(19) United States
(12) Patent Application Publication
(10) Pub. No.: US 2013/0131826 A1
(43) Pub. Date: May 23, 2013

(54) ARTIFICIAL BONE-CARTILAGE COMPOSITE AND ITS PRODUCTION METHOD

(71) Applicant: HOYA CORPORATION, Tokyo (JP)

(72) Inventors: Machiko KATO, Tokyo (JP); Tomoji TAKAYAMA, Tokyo (JP); Yuko KOZAKA, Tokyo (JP)

(73) Assignee: HOYA CORPORATION, Tokyo (JP)

(21) Appl. No.: 13/670,715

(22) Filed: Nov. 7, 2012

(30) Foreign Application Priority Data
Nov. 11, 2011 (JP) JP2011-248076

(51) Int. Cl. A61F 2/28

(52) U.S. Cl. CPC A61F 2/28
USPC 623/23.63

(57) ABSTRACT

An artificial bone-cartilage composite comprising a first composite material layer comprising collagen, proteoglycan and hyaluronic acid, and a second composite material layer comprising collagen and calcium phosphate, which are bonded to each other via a bonding layer comprising collagen, proteoglycan, hyaluronic acid and calcium phosphate.

### FIELD OF THE INVENTION

The present invention relates to an artificial bone-cartilage composite comprising a highly elastic artificial cartilage derived from living cartilage components and an artificial bone, which are strongly bonded to each other, and its production method.

### BACKGROUND OF THE INVENTION

Tissues are destroyed with time in articular bone defects such as osteoarthritis, articular rheumatism, etc., and articular cartilage tissues are extremely poor in reparability. Accordingly, in addition to pharmacological treatments, replacement arthroplasty has become widely used for the treatment of articular bone diseases. Artificial bones comprising hydroxyapatite as a main component, which were developed for the treatment of bone diseases and degenerations, have high affinity for bones and rigidity similar to that of bones, exhibiting good treatment results when implanted or embedded in bone defects in bone diseases such as bone fracture, bone tumor, etc.

Replacement arthroplasty with a metal is conducted on extremely deformed or and degenerated cartilage surfaces of articular tissues, and technologies of regenerating cartilage resembling the living cartilage in both structure and function have recently been developed. In diseases accompanied by extreme deformation and degeneration of articular bones, damage exists not only in cartilage tissues but also in subchondral bones and bone tissues near joints in many cases, making it necessary to regenerate cartilages and subchondral bones.

US 2009/0311221 A1 discloses a method for producing a self-organized composite of glycosaminoglycan, proteoglycan and collagen comprising the steps of (a) mixing glycosaminoglycan with proteoglycan to prepare a glycosaminoglycan-proteoglycan aggregate, and (b) mixing the glycosaminoglycan-proteoglycan aggregate with collagen.

U.S. Pat. No. 7,153,938 B2 discloses a method for producing a porous, cross-linked apatite/collagen composite, which is absorbed by the body by the same mechanism as that of the living bone and has such high osteogenesis that it can be used for artificial bone, etc., the method comprising gelling a dispersion comprising an apatite/collagen composite and collagen, freeze-drying the gelled dispersion to produce a porous body, and cross-linking collagen in the porous body.

JP 2009-268494 A discloses a method for producing an artificial bone/cartilage biomaterial, in which a cartilage-like composite is integrally fused to an artificial bone, the method comprising the steps of contacting a cartilage-like composite comprising glycosaminoglycan, proteoglycan and collagen with an artificial bone, and self-organizing said cartilage-like composite on the artificial bone. JP 2009-268494 A describes that by the self-organization of the cartilage-like composite on the artificial bone, collagen fibers are bonded to not only an artificial bone surface of porous hydroxyapatite but also pores in the artificial bone. In the biomaterial produced by the method described in JP 2009-268494 A, however, the cartilage-like composite merely intrudes pores of the artificial bone, but may be peeled from the artificial bone. Thus, further improvement is desired.

JP 2011-36320 A discloses an implanting multilayer cartilage block, which is constituted by a laminate comprising a low-porosity, porous gelatin layer and a high-porosity, porous gelatin layer; blood flows into said low-porosity, porous gelatin layer being restricted, and cartilage cells enter the high-porosity, porous gelatin layer, thereby accelerating the growth of cartilage cells for rapid repair of cartilage defects. However, the implanting multilayer cartilage block described in JP 2011-36320 A is constituted by two porous gelatin layers having different porosities, failing to have the functions of a composite material comprising an artificial cartilage bonded to an artificial bone.

US 2009/0022771 A1 discloses a method for producing a composite biomaterial comprising an inorganic material and an organic material, the method comprising the steps of (1) preparing a first slurry composition containing a liquid carrier, an inorganic material and an organic material; (2) preparing a second slurry composition containing a liquid carrier, an organic material and optionally an inorganic material; (3) charging said second slurry composition into a mold before or after charging said first slurry composition into the mold; (4) cooling the slurries in the mold at a temperature at which the liquid carrier is turned to pluralities of solid crystals or particles; (5) removing at least part of the solid crystals or particles preferably by sublimation and/or evaporation, to provide a porous composite material containing the inorganic material and the organic material; and (6) removing the porous composite material from the mold. US 2009/0022771 A1 describes that said first slurry composition comprises a three-component coprecipitate of collagen, calcium phosphate and glycosaminoglycan, and that said second slurry composition comprises a two-component coprecipitate of collagen and glycosaminoglycan, a two-component coprecipitate of collagen and calcium phosphate, or a three-component coprecipitate of collagen, glycosaminoglycan and calcium phosphate. However, because two different compositions in the form of slurry are laminated by the method of US 2009/0022771 A1, the resultant composite material is a substantially uniform mixture of two slurries, failing to have a structure in which an artificial cartilage is bonded to an artificial bone.

### OBJECT OF THE INVENTION

Accordingly, an object of the present invention is to provide an artificial bone-cartilage composite comprising an artificial cartilage strongly bonded to an artificial bone, and its production method.

### DISCLOSURE OF THE INVENTION

As a result of intensive research in view of the above object, the inventors have found that the bonding of a first composite material layer comprising collagen, proteoglycan and hyaluronic acid to a second composite material layer comprising collagen and calcium phosphate via a bonding layer composed of a mixture of the first and second materials provides a laminate comprising said first composite material layer and said second composite material layer extremely strongly bonded to each other, which is suitable as an artificial bone-cartilage composite. The present invention has been completed based on such finding.

Thus, the artificial bone-cartilage composite of the present invention comprises a first composite material layer comprising collagen, proteoglycan and hyaluronic acid, and a second composite material layer comprising collagen and calcium phosphate, which are bonded to each other via a bonding layer comprising collagen, proteoglycan, hyaluronic acid and calcium phosphate.

Said first composite material layer preferably comprises 15-95% by mass of collagen, 4.9-70% by mass of proteoglycan, and 0.1-20% by mass of hyaluronic acid.

Said second composite material layer is preferably a porous apatite/collagen composite.

An apatite/collagen ratio in said second composite material layer is preferably 8/2-2/8 by mass.

The artificial bone-cartilage composite of the present invention is preferably cross-linked.

The method of the present invention for producing a first composite material layer comprising collagen, proteoglycan and hyaluronic acid, and a second composite material layer comprising collagen and calcium phosphate, which are bonded to each other via a bonding layer comprising collagen, proteoglycan, hyaluronic acid and calcium phosphate comprises the steps of (1) preparing a first composite material comprising collagen, proteoglycan and hyaluronic acid, (2) preparing a second composite material comprising collagen and calcium phosphate, (3) laminating said first composite material and said second composite material, and (4) freeze-drying the resultant laminate.

Said first composite material and/or said second composite material are preferably subject to a centrifugal treatment before or after the laminating step.

Said laminate is preferably gelled before the freeze-drying step.

Said first composite material is preferably in the form of gel, and said second composite material is preferably in the form of slurry or gel.

### DESCRIPTION OF THE PREFERRED EMBODIMENTS

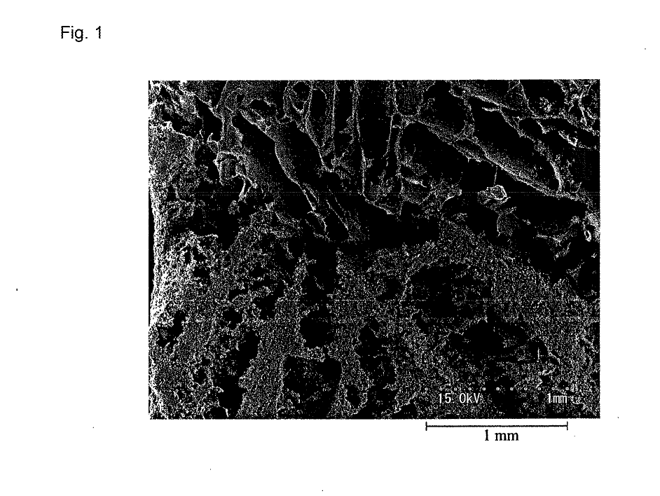

FIG. 1 is an electron photomicrograph showing a boundary portion of an artificial cartilage and an artificial bone in the artificial bone-cartilage composite of the present invention.

### DESCRIPTION OF THE PREFERRED EMBODIMENTS

[1] Artificial Bone-Cartilage Composite

(1) Entire Structure

The artificial bone-cartilage composite of the present invention comprises a first composite material layer comprising collagen, proteoglycan and hyaluronic acid, and a second composite material layer comprising collagen and calcium phosphate, which are bonded to each other via a bonding layer comprising collagen, proteoglycan, hyaluronic acid and calcium phosphate. The first composite material layer functions as a so-called artificial cartilage, and the second composite material layer functions as a so-called artificial bone.

Said bonding layer has a gradually changing composition by the mutual diffusion of said first material and the second material, so that it does not have clear boundaries. A portion in which said first material and the second material are mixed by diffusion are called “bonding layer” herein.

Accordingly, said bonding layer has a mixed composition of said first material and the second material, namely a composition comprising collagen, proteoglycan, hyaluronic acid and calcium phosphate. The composition of the bonding layer becomes closer to that of the first material comprising collagen, proteoglycan and hyaluronic acid as the bonding layer goes closer to said first composite material layer, and closer to that of the second composite material comprising collagen and calcium phosphate as the bonding layer goes closer to said second composite material layer.

With such a bonding layer in which said first material and the second material are mixed, said first composite material layer and the second composite material layer are so strongly bonded to each other that they are not easily peeled when implanted in defects.

The size and shape of the artificial bone-cartilage composite, and a dimensional ratio of the first composite material layer to the second composite material layer may be properly set depending on sites in which the artificial bone-cartilage composite is used. In general, said first composite material layer is preferably as thick as about 1 mm to about 2 cm, and said second composite material layer is preferably as thick as about 5 mm to about 10 cm. They may have circular or rectangular shapes, though not restrictive. Said first composite material layer and said second composite material layer may have the same or different shapes.

(B) First Composite Material Layer

The first composite material layer comprises collagen, proteoglycan and hyaluronic acid, and functions as a so-called artificial cartilage. The first composite material layer preferably comprises 15-95% by mass of collagen, 4.9-70% by mass of proteoglycan, and 0.1-20% by mass of hyaluronic acid. Collagen forms a network structure acting as a skeleton for cartilage tissues, and its physical and/or chemical cross-linking with hyaluronic acid and proteoglycan makes it possible to retain sufficient water, providing an artificial cartilage (first composite material layer) having elasticity peculiar to the cartilage. The amounts of collagen, proteoglycan and hyaluronic acid in the first composite material layer are more preferably 45-65% by mass, 20-40% by mass and 1.5-5% by mass, respectively. Within this range, the artificial cartilage is particularly suitable as an articular cartilage.

When the collagen content is less than 15% by mass, the artificial cartilage exhibits a large expansion ratio when inserted into the body, so that it cannot be easily fitted to cartilage defects. In addition, the expansion reduces the porosity of the artificial cartilage. When the collagen content is more than 95% by mass, the artificial cartilage is extremely colored. When the proteoglycan content is less than 4.9% by mass, the artificial cartilage has low elasticity, poor performance as cartilage. When the proteoglycan content is more than 70% by mass, the artificial cartilage suffers large size change due to expansion, resulting in low porosity. When the hyaluronic acid content is less than 0.1% by mass, the artificial cartilage has low elasticity, poor performance as cartilage, and low surface lubrication (losing low-friction characteristics). More than 20% by mass of hyaluronic acid largely exceeds its percentage in the living cartilage, making the artificial cartilage different from the living cartilage, resulting in difficulty to secure a desired ratio of collagen to proteoglycan depending on portions in which the artificial cartilage is used.

The collagen is not particularly restricted, but may be extracted from animals, etc. Animals from which collagen is obtained are not particularly restricted in types, tissues, ages, etc. Generally usable are collagen obtained from skins, bones, cartilages, tendons, internal organs, etc. of mammals such as cow, pig, horse, rabbit and rat, and birds such as hen, etc. Collagen-like proteins obtained from skins, bones, cartilages, fins, scales, internal organs, etc. of fish such as cod, flounder, flatfish, salmon, trout, tuna, mackerel, red snapper, sardine, shark, etc. may also be used. The extraction method of collagen is not particularly restrictive but may be a usual one. In place of collagen extracted from animal tissues, synthesized collagen and those produced by gene recombination technologies may also be used.

Glycosaminoglycan is a kind of acidic polysaccharides having a repeating disaccharide unit comprising aminosugar combined with uronic acid or galactose. Hyaluronic acid used in the present invention is a kind of glycosaminoglycans. Though other glycosaminoglycans than hyaluronic acid, such as chondroitin sulfate, dermatan sulfate, heparan sulfate, keratan sulfate, heparin, etc. are usable, it is preferable to use hyaluronic acid.

The proteoglycan is a compound having one or more glycosaminoglycan chains bonded to one protein acting as a nucleus. The proteoglycan is not particularly restricted, but may be aggrecan, versican, neurocan, brevican, decorin, biglycan, serglycin, perlecan, syndecan, glypican, lumican, keratocan, etc. Among them, aggrecan is preferable.

Proteoglycan sources are not particularly restricted, and various animals such as mammals (humans, cow, pig, etc.), birds (hen, etc.), fish (shark, salmon, etc.), crustaceans (crabs, shrimps, etc.), etc. may be properly used, depending on the applications of the composite. Particularly when the artificial bone-cartilage composite of the present invention is used for curing human cartilage defects or degenerations, it is preferable to select those having low human immunogenicity.

The determination of collagen in the artificial cartilage can be conducted by a UV absorption measurement method, an HPLC method, etc. The determination of hyaluronic acid can be conducted by a carbazole-sulfuric acid method, an inhibition method using a hyaluronic-acid-binding protein, an HPLC method, etc. The determination of proteoglycan can be conducted by a colorimetric determination method using a pigment DMMB, an HPLC method, etc.

The artificial cartilage is preferably cross-linked. The cross-linking treatment can be conducted by a physical or chemical method. The artificial cartilage is also preferably sterilized by such a method as a γ-ray irradiation method, etc.

The porosity of the artificial cartilage is preferably 50-99%, more preferably 60-99%. The average pore diameter of the artificial cartilage is preferably 1-1000 μm, more preferably 50-800 μm.

(C) Second Composite Material Layer

The second composite material layer comprises collagen and calcium phosphate, and functions as a so-called artificial bone. The second composite material layer is preferably a porous apatite/collagen composite. The porous apatite/collagen composite becomes a deformable sponge-like body when wetted, easily applicable to any sites having complicated shapes in patients. The second composite material layer is substituted by an autogenous bone with time.

The porous apatite/collagen composite is constituted by pluralities of layers of apatite/collagen composite fibers. The fiber layers have planar shapes as thick as about 10-500 μm and overlapping in random directions with random numbers. Disposed sparsely between the fiber layers are pillars composed of the apatite/collagen composite fibers. Because only sparsely arranged pillars support the fiber layers in an overlapping direction when viewed microscopically, it may be considered that the porous apatite/collagen composite is relatively brittle in the overlapping direction, while it has high strength in a layer direction. However, because the fiber layers are overlapping in random directions as described above, the overlapping directions of the fiber layers are averaged when viewed macroscopically, resulting in little anisotropy of strength.

Substantially planar-shaped pores are formed between the fiber layers with pillars. The thickness of the substantially planar-shaped pores is about 0.5-10 times that of the fiber layers. When this porous apatite/collagen composite is embedded in the body, it is considered that blood vessels, relatively large proteins, etc. easily enter substantially planar pores, accelerating bone formation.

An apatite/collagen ratio in the porous apatite/collagen composite is preferably 8/2-2/8 by mass, particularly about 8/2 by mass. The porosity of the porous apatite/collagen composite is preferably 70-99%, more preferably 80-97%. The average pore diameter of the porous apatite/collagen composite is preferably 1-1000 μm, more preferably 100-700 μm.

(D) Bonding Layer

The boundary portion of said first composite material layer and the second composite material layer is constituted by a bonding layer comprising collagen, proteoglycan, hyaluronic acid and calcium phosphate. As shown in FIG. 1, said bonding layer has a composition obtained by combining said first material with the second material, thereby strongly bonding both composite material layers. The bonding layer is preferably as thick as having practical strength to prevent said first and second composite material layers from peeling. Specifically, the thickness of the bonding layer is preferably 1-3000 μm, more preferably 1-2000 μm, most preferably 1-1000 μm.

[2] Production Method

The method of the present invention for producing the artificial bone-cartilage composite comprises the steps of (1) preparing a first composite material comprising collagen, proteoglycan and hyaluronic acid, (2) preparing a second composite material comprising collagen and calcium phosphate, (3) laminating said first composite material with the second composite material, and (4) freeze-drying the resultant laminate.

Said first composite material and/or said second composite material are preferably subject to a centrifugal treatment before or after the laminating step. Said laminate is preferably gelled before the freeze-drying step. Said first composite material is preferably in the form of gel, and said second composite material is preferably in the form of slurry or gel.

(A) Preparation of First Composite Material

The first composite material is obtained by mixing collagen, proteoglycan and hyaluronic acid, starting materials for artificial cartilage, in such proportions as to provide a composition comprising 15-95% by mass of collagen, 4.9-70% by mass of proteoglycan and 0.1-20% by mass of hyaluronic acid. The collagen is preferably dissolved in water or dilute hydrochloric acid (concentration: about 5-50 mM) in a concentration of 0.1-20% by mass in advance. The proteoglycan is preferably dissolved in sterile water (water for injection, etc.) in a concentration of 0.1-20% by mass in advance. The hyaluronic acid is preferably dissolved in sterile water (water for injection, etc.) in a concentration of 0.1-20% by mass in advance.

The first composite material can be prepared by conducting (a-1) a first mixing method in which a mixture of hyaluronic acid and collagen and a mixture of proteoglycan and collagen are separately prepared and then mixed, or (a-2) a second mixing method in which collagen, proteoglycan and hyaluronic acid are simultaneously mixed, and then (b) freeze-drying the resultant mixture (first freeze-drying). After the first freeze-drying step, a step of pulverizing the freeze-dried product, a step of dispersing the pulverized, freeze-dried product in water, and a step of freeze-drying the resultant dispersion again (second freeze-drying step) may be conducted.

(1-1) First Mixing Method

The first mixing method comprises the steps of (a) preparing a first composition comprising hyaluronic acid and collagen, (b) preparing a second composition comprising proteoglycan and collagen, and (c) mixing the first composition with the second composition.

(a) Preparation of First and Second Compositions

In the step of preparing the first composition, an aqueous hyaluronic acid solution and an aqueous collagen solution are mixed, such that a mixing ratio (by mass) of hyaluronic acid to collagen is preferably 10000/1 to 1/10000, more preferably 5000/1 to 1/5000, most preferably 15/1 to 1/15. The aqueous hyaluronic acid solution and the aqueous collagen solution are preferably mixed at 3° C. to 25° C.

In the step of preparing the second composition, an aqueous proteoglycan solution and an aqueous collagen solution are mixed, such that a mixing ratio (by mass) of proteoglycan to collagen is preferably 10000/1 to 1/10000, more preferably 5000/1 to 1/5000, most preferably 10/1 to 1/10. The aqueous proteoglycan solution and the aqueous collagen solution are preferably mixed at 3° C. to 25° C.

Because the mixing of the aqueous hyaluronic acid solution and the aqueous collagen solution (the preparation of the first composition) and the mixing of the aqueous proteoglycan solution and the aqueous collagen solution (the preparation of the second composition) do not need particularly high shearing, usual apparatuses such as stirrers, mixers, etc. may be used. The mixing is preferably conducted at 3° C. to 25° C. for about 1 second to about 3 minutes, to obtain a homogeneous mixture of hyaluronic acid and collagen, and a homogeneous mixture of proteoglycan and collagen, respectively.

(b) Mixing of First and Second Compositions

The mixing ratio of the first composition to the second composition is determined such that the resultant mixture comprises 15-95% by mass of collagen, 4.9-70% by mass of proteoglycan, and 0.1-20% by mass of hyaluronic acid. The first composition is mixed with the second composition preferably by a method using a shearing force by a homogenizer, a dissolver, etc. For example, when the homogenizer is used, a stirring step at 1,000 to 12,000 rpm for 30 seconds to 3 minutes is preferably repeated 2 to 5 times. During mixing, a sample is preferably kept at about 3° C. to about 25° C. The mixing of the first and second compositions, which are separately prepared, accelerates the synthesis of cartilage.

(1-2) Second Mixing Method

In the second mixing method, an aqueous collagen solution, an aqueous proteoglycan solution and an aqueous hyaluronic acid solution are simultaneously mixed, such that the resultant composition comprises 15-95% by mass of collagen, 4.9-70% by mass of proteoglycan and 0.1-20% by mass of hyaluronic acid.

The aqueous collagen solution, the aqueous proteoglycan solution and the aqueous hyaluronic acid solution are preferably mixed under a shearing force using an apparatus such as a homogenizer, a dissolver, etc. For example, when the homogenizer is used, stirring at 1,000 to 12,000 rpm for 30 seconds to 3 minutes is preferably repeated 2 to 5 times. The mixing of the aqueous collagen solution, the aqueous proteoglycan solution and the aqueous hyaluronic acid solution are preferably conducted at 3° C. to 25° C.

(2) First Freeze-Drying

A mixture obtained by the first mixing method or the second mixing method is cast into a heat-conductive vessel (metal tray, etc.), and frozen at −80° C. to −60° C. overnight. The frozen mixture is subject to a first drying step at a shelf temperature of about −50° C. to about −5° C. (preferably −40° C. to −5° C.) in vacuum for about 10 hours to about 10 days until the mixture loses water (ice) substantially completely, and then a second drying step at a shelf temperature of about 20° C. to about 40° C. (preferably 25° C. to 40° C.) in vacuum for 3 to 24 hours. Even bound water can be removed by such two-step freeze-drying at different temperatures, providing a well freeze-dried product having excellent storability.

(3) Pulverization

The freeze-dried product is pulverized by a solid-pulverizing apparatus such as a mill, etc. A pulverization method is not particularly restricted, but preferably a method not exposing the freeze-dried product to an excessively high temperature.

(4) Dispersion

The pulverized, freeze-dried product is mixed with water or a physiological saline to a concentration of 3-20% by mass, and subject to a dispersion treatment at 3° C. to 25° C. and at 1,000 to 15,000 rpm for 30 seconds to 3 minutes 1 to 5 times using an apparatus such as a homogenizer, etc., thereby obtaining the first composite material.

(5) Gelation

The dispersion (the first composite material) may be gelled if necessary. The gelation is preferably conducted by casting the first composite material into a vessel such as a culture dish, etc. and covering it, and then leaving it to stand at 30° C. to 40° C. for 1 to 5 hours.

(B) Preparation of Second Composite Material

(1) Composition

The second composite material is a slurry containing apatite/collagen composite fibers, which are obtained from collagen, phosphoric acid or its salt, and a calcium salt as starting materials. The collagen is not particularly restricted, but may be extracted from animals, etc. The kinds, parts, ages, etc. of the animals are not particularly restrictive. In general, collagen obtained from skins, bones, cartilages, tendons, internal organs, etc. of mammals such as cow, pig, horse, rabbit and rat, and birds such as hen, etc. may be used. Collagen-like proteins obtained from skins, bones, cartilages, fins, scales, internal organs, etc. of fish such as cod, flounder, flatfish, salmon, trout, tuna, mackerel, red snapper, sardine, shark, etc. may also be used. The extraction method of collagen is not particularly restrictive but may be a usual one. In place of collagen extracted from animal tissues, synthesized collagen and those produced by gene recombination technologies may also be used.

Phosphoric acid or its salts [hereinafter referred to simply as “phosphoric acid (salt)”] include phosphoric acid, disodium hydrogenphosphate, sodium dihydrogenphosphate, dipotassium hydrogenphosphate, and potassium dihydrogenphosphate. The calcium salts include calcium carbonate, calcium acetate, and calcium hydroxide. The phosphate and the calcium salt are preferably added in the form of a uniform aqueous solution or suspension.

By a mass ratio of the apatite-forming materials [phosphoric acid (salt) and calcium salts] to the collagen, an apatite/collagen mass ratio in the second composite material can be controlled. Accordingly, the mass ratio of the apatite-forming materials used to the collagen may be determined properly depending on the target proportion of the apatite/collagen composite fibers. The mass ratio of the apatite-forming materials to the collagen is preferably 9/1-6/4, particularly about 8/2.

(2) Preparation

An aqueous solution of collagen and phosphoric acid (salt), which is simply called “an aqueous collagen/phosphoric acid (salt) solution,” and an aqueous solution or suspension of a calcium salt are prepared. In the aqueous collagen/phosphoric acid (salt) solution, the concentration of collagen is preferably 0.1-1.2% by mass, particularly about 0.85% by mass, and the concentration of the phosphoric acid (salt) is preferably about 50-250 mM. The concentration of the aqueous calcium salt solution (or suspension) is about 200 mM to about 600 mM. It is preferable to add the collagen in the form of a low-concentration, aqueous phosphoric acid solution to the aqueous solution of phosphoric acid (salt), to prepare the aqueous collagen/phosphoric acid (salt) solution. In the aqueous collagen/phosphoric acid (salt) solution, the concentration of collagen is preferably 0.5-1.5% by mass, and the concentration of phosphoric acid is preferably 10-30 mM.

(3) Synthesis

The aqueous collagen/phosphoric acid (salt) solution and the aqueous calcium salt solution (or suspension) are simultaneously dropped into water at about 40° C., whose amount is preferably 0.5-2 times, more preferably 0.8-1.2 times, particularly substantially the same as that of the aqueous calcium salt solution (or suspension), to synthesize apatite/collagen composite fibers. The length of the apatite/collagen composite fibers can be controlled by the dropping conditions such as a temperature, a dropping speed, a stirring speed, etc. The dropping speed is preferably about 10-50 ml/minute, and the reaction solution is preferably stirred at about 50-300 rpm.

During the dropping, the reaction solution is preferably kept at pH of 8.9-9.1, to keep the concentrations of calcium ions and phosphoric acid ions to 3.75 mM or less and 2.25 mM or less, respectively. When the concentrations of calcium ions and/or phosphoric acid ions are higher than the above ranges, the apatite/collagen composite is not self-organized. The term “self-organization” used herein means that hydroxyapatite (calcium phosphate having an apatite structure) has orientation peculiar to the living bone along collagen fibers, namely that the C-axis of the hydroxyapatite is in alignment with the collagen fibers. Under the above dropping conditions, the apatite/collagen composite fibers are self-organized to a length of 1 mm or less suitable for the porous body.

After the completion of dropping, a slurry-like dispersion containing the apatite/collagen composite fibers is freeze-dried. The freeze-drying can be carried out by rapid drying in vacuum in a frozen state at −10° C. or lower.

(4) Preparation of dispersion

The apatite/collagen composite fibers are mixed with water, an aqueous phosphoric acid solution, etc. and stirred to prepare a paste-like dispersion. The amount of a liquid contained in this dispersion is preferably 80-99% by volume, more preferably 90-97% by volume. Namely, the amount of the composite fibers is preferably 1-20% by volume, more preferably 3-10% by volume. Steam is preferably attached to the apatite/collagen composite fibers in advance. In this case, the amount of water to be added should be determined with the amount of steam attached to the apatite/collagen composite fibers subtracted.

The resultant porous body has porosity P (%), which depends on a volume ratio of the apatite/collagen composite fibers to the liquid in the dispersion, as represented by the following formula (1):

P=Y/(X+Y)×100 (1),

wherein X represents the volume of the apatite/collagen composite fibers in the dispersion, and Y represents the volume of the liquid in the dispersion. Accordingly, it is possible to control the porosity P of the porous body by adjusting the amount of the liquid to be added. The apatite/collagen composite fibers are cut to have a wide length distribution by stirring the dispersion after adding the liquid, resulting in a porous body with improved strength.

Collagen as a binder is added to the dispersion of apatite/collagen composite fibers, and further stirred. The amount of the collagen added is preferably 1-10% by mass, more preferably 3-6% by mass, based on 100% by mass of the apatite/collagen composite fibers. As in the case of the apatite/collagen composite fibers, the collagen is added preferably in the form of an aqueous phosphoric acid solution. Though the concentrations of collagen and phosphoric acid in the aqueous collagen/phosphoric acid (salt) solution are not particularly restricted, it is practically preferable that the concentration of collagen is 0.8-0.9% by mass (for instance, 0.85% by mass), and that the concentration of phosphoric acid is 15-25 mM (for instance, 20 mM).

The dispersion turned acidic by the addition of the aqueous collagen/phosphoric acid (salt) solution is mixed with a sodium hydroxide solution to adjust its pH to preferably 6.8-7.6, more preferably 7.0-7.4, particularly about 7. By adjusting the pH of the dispersion to 6.8-7.6, the collagen added as a binder is quickly turned to fibers.

The phosphate buffer solution (PBS) as concentrated as about 2.5-10 times is preferably added to the dispersion and stirred, to adjust its ion strength as high as that of PBS (about 0.2-0.8). The larger ion strength of the dispersion accelerates the collagen added as a binder to be turned to fibers.

Thus obtained is the second composite material in the form of an apatite/collagen composite slurry. Incidentally, any one or all of the addition of a binder, the adjustment of pH, and the adjustment of ion strength may be omitted. The apatite/collagen composite slurry may be kept at 35-43° C. for 0.5-3.5 hours for gelation.

(C) Lamination of First and Second Composite Materials

The first composite material gel and the second composite material slurry or gel are laminated in a vessel without disturbing their boundary. The order of laminating the first composite material and the second composite material is not particularly restricted, but it is preferable to place the second composite material in a vessel first, and then laminate the first composite material on the second composite material without disturbing their boundary.

When both composite materials are placed in a vessel successively, a centrifugal separation treatment is preferably conducted to obtain a flat boundary. For example, with one composite material constituting a lower layer placed in a vessel, a first centrifugal separation treatment is conducted to provide one composite material with a flat surface. Next, the other composite material constituting an upper layer is charged into the vessel to laminate them, and a second centrifugal separation treatment is conducted in this state. Thus obtained is a laminate with a flat boundary portion between the two composite material layers. One of the first centrifugal separation treatment and the second centrifugal separation treatment may be omitted.

(D) Gelation

By keeping the resultant laminate at a predetermined temperature, the laminate is gelled, and the components constituting the first and second composite materials (collagen, proteoglycan, hyaluronic acid and calcium phosphate) are diffused mutually. Thus obtained is a bonding layer having a composition comprising collagen, proteoglycan, hyaluronic acid and calcium phosphate, the composition changing continuously such that it contains more proteoglycan and hyaluronic acid (it becomes closer to the composition of the first composite material layer) as nearing the first composite material layer, and that it contains more calcium phosphate (it becomes closer to the composition of the second composite material layer) as nearing the second composite material layer. The temperature kept for gelation and the formation of the bonding layer is preferably 35° C. to 43° C., more preferably 35° C. to 40° C. To achieve the full gelation of the dispersion to form the bonding layer, the temperature-keeping time is preferably 0.5-3.5 hours, more preferably 1-3 hours.

(E) Second Freeze-Drying

The gelled laminate is frozen and dried to obtain the artificial bone-cartilage composite. During freeze-drying, a vessel containing the gelled laminate is preferably placed on a net plate in a stainless steel tray. Freezing is preferably conducted after cooling the gelled laminate at 2° C. to 10° C. for 1-24 hours. The freezing temperature is preferably −100° C. to 0° C., more preferably −90° C. to −10° C., most preferably −80° C. to −20° C. When the freezing temperature is lower than −100° C., the freeze-dried, porous apatite/collagen composite layer (the second composite material layer) has too small pores. Freezing does not occur at a temperature higher than 0° C. The freezing time is preferably 12-48 hours.

The frozen laminate is subject to a first drying step at a shelf temperature of about −50° C. to about −5° C. (preferably −40° C. to −5° C.) in vacuum for about 3 days to about 1 week until the laminate loses water (ice) substantially completely, and then to a second drying step at a shelf temperature of about 20° C. to about 40° C. (preferably 25° C. to 40° C.) in vacuum for 3 to 24 hours. Even bound water can be removed by such two-step freeze-drying at different temperatures, providing a well freeze-dried product having excellent storability.

(F) Cross-Linking and Sterilization Treatment

To provide the artificial bone-cartilage composite with increased mechanical strength and high long-period retainability when inserted into the body, the freeze-dried, porous laminate is preferably cross-linked. The cross-linking treatment can be conducted by physical cross-linking methods using γ-rays, ultraviolet rays, electron beams, thermal dehydration, etc., or chemical cross-linking methods using cross-linking agents, condensation agents, etc. The chemical cross-linking methods include, for example, a method of immersing the freeze-dried, porous laminate in a cross-linking agent solution, a method of applying a steam containing a cross-linking agent to the freeze-dried, porous laminate, and a method of adding a cross-linking agent to aqueous solutions or dispersions of the first and second composite materials.

Among these methods, the thermal dehydration cross-linking method is preferable in the present invention. The thermal dehydration cross-linking can be conducted by keeping the freeze-dried, porous laminate in a vacuum oven at 100° C. to 160° C. and 0 to 100 hPa for 10 to 30 hours.

The artificial bone-cartilage composite thus obtained is preferably sterilized by ultraviolet rays, γ-rays, electron beams, drying by heat, etc. Particularly preferable sterilization is the irradiation of γ-rays of 25 kGy or less.

The present invention will be explained in more detail referring to Examples, without intention of restricting the present invention to them.

### EXAMPLE 1

(1) Preparation of First Composite Material

Collagen was dissolved in 5-mM hydrochloric acid to prepare an aqueous collagen solution having a concentration of 1% by mass. Proteoglycan was dissolved in water for injection to prepare an aqueous proteoglycan solution having a concentration of 1% by mass. Hyaluronic acid was dissolved in water for injection to prepare an aqueous hyaluronic acid solution having a concentration of 0.1% by mass. All of these preparation steps were conducted at 4° C.

The aqueous collagen solution was mixed with the aqueous proteoglycan solution at a mass ratio of 1/1, and stirred by a mixer to obtain a mixture solution A. Likewise, the aqueous collagen solution was mixed with the aqueous hyaluronic acid solution at a mass ratio of 1/1, and stirred by a mixer to obtain a mixture solution B. The mixture solutions A and B were mixed at a mass ratio of 2/1, and subject to stirring by a homogenizer at 10,000 rpm for 1 minute 3 times with 30-seconds intervals. The stirring was conducted while the temperature of a sample was kept at 5° C.

The resultant mixture was subject to the first freeze-drying step by the following procedures. Namely, the mixture was cast into a tray, frozen at −80° C. for 19 hours, and then subject to first drying at a shelf temperature of −5° C. in vacuum for 10 days. By the first drying, the mixture lost substantially all water (ice). While continuing evacuation, second drying was then conducted at a shelf temperature of 25° C. for 3 hours, thereby obtaining a freeze-dried product.

The freeze-dried product was pulverized by a mill, and the pulverized, freeze-dried product was mixed with a physiological saline to a concentration of 10.7% by mass, and subject to a dispersion treatment at 10,000 rpm for 1 minute by a homogenizer 3 times with one-minute intervals. During dispersion by the homogenizer, the mixture was kept at 5° C.

The resultant dispersion was stirred for 1 minute by a planetary centrifugal mixer (ARE-250 available from Thinky Corporation), to remove bubbles from the dispersion.

(2) Preparation of Second Composite Material

350 g of an aqueous collagen/phosphoric acid solution (collagen concentration: 0.57% by mass, and phosphoric acid concentration: 20 mM) was added to 42 ml of an 120-mM aqueous phosphoric acid solution, and stirred to prepare an aqueous collagen/phosphoric acid solution called “liquid I.” Also, 200 ml of a 400-mM aqueous calcium hydroxide suspension called “liquid II” was prepared. 200 ml of pure water was charged into a reaction vessel and heated to 38° C. The collagen/phosphoric acid aqueous solution (liquid I) and the aqueous calcium hydroxide suspension (liquid II) were simultaneously dropped into pure water in this reaction vessel both at a speed of about 30 ml/minute, while stirring the resultant reaction solution at 200 rpm, thereby preparing a slurry containing apatite/collagen composite fibers. The dropping speed was controlled such that the pH of the reaction solution was kept at 8.9-9.1. The slurry containing apatite/collagen composite fibers was freeze-dried at −30° C. The resultant apatite/collagen composite fibers were mostly as long as 1 mm or less, having an apatite/collagen mass ratio of 8/2.

2 parts by mass of the freeze-dried apatite/collagen composite fibers were mixed with 13 parts by mass of physiological saline and stirred to prepare a paste-like dispersion. The dispersion was left to stand at 37.5° C. for 2 hours, and then at 5° C. for gelation, thereby obtaining a gel-like apatite/collagen composite material (second composite material).

(3) Production of Laminate

3 g of the gel-like apatite/collagen composite material (second composite material) was charged into a culture multiplate (volume: 3.4 mL), and subject to a centrifugal separation treatment at 3,000 rpm (about 1200 G) and 4° C. for 5 minutes. The gel-like collagen/proteoglycan/hyaluronic acid composite material (first composite material) was slowly put on the centrifugally treated second composite material, and subject to a centrifugal separation treatment at 3,000 rpm (about 1200 G) and 4° C. for 1 minute.

(4) Gelation and Cooling

The resultant laminate was left to stand at 37.5° C. for 1 hour for gelation, and then cooled at 5° C. for 2 hours.

(5) Second Freeze-Drying

The cooled laminate in the vessel was placed on a net plate in a stainless steel tray, frozen at −60° C. for 17 hours, and then subject to first drying at a shelf temperature of −5° C. for 6 days in vacuum. By this first drying, the laminate lost water (ice) substantially completely. Second drying was then conducted at a shelf temperature of 25° C. in vacuum for 4 hours to obtain a freeze-dried product.

(6) Cross-Linking and Sterilization

The freeze-dried product was subject to thermal dehydration cross-linking at 110° C. for 20 hours in a vacuum oven, and then to a sterilization treatment with γ-rays of 25 kGy, thereby obtaining an artificial bone-cartilage composite comprising a first composite material layer comprising collagen, proteoglycan and hyaluronic acid, which was laminated on a second composite material layer comprising collagen and calcium phosphate.

FIG. 1 is an electron photomicrograph showing a boundary portion (bonding layer) between the first and the second composite material layers in the artificial bone-cartilage composite. FIG. 1 shows the first composite material layer functioning as an artificial cartilage on an upper side, and the second composite material layer functioning as an artificial bone on a lower side. Because this boundary portion does not have clear interfaces, it is presumed that the first and second composite materials are mutually diffused, forming a bonding layer comprising collagen, proteoglycan, hyaluronic acid and calcium phosphate. It was further observed that other portions than the boundary portion were occupied by the first or second composite material alone.

When the artificial bone-cartilage composite was immersed in water and then evacuated, the first and second composite material layers did not peel in the boundary portion, indicating that the first and second composite material layers were strongly bonded to each other.

### EFFECT OF THE INVENTION

Because the artificial bone-cartilage composite of the present invention has a first composite material (artificial cartilage) layer comprising collagen, proteoglycan and hyaluronic acid, and a second composite material (artificial bone) layer comprising collagen and calcium phosphate, which are strongly bonded to each other, it is particularly suitable as an artificial bone-cartilage composite for filling defects which may receive high pressure. The method of the present invention can easily and surely produce an artificial bone-cartilage composite comprising an artificial cartilage and an artificial bone bonded to each other with high strength.

What is claimed is:

1. An artificial bone-cartilage composite comprising a first composite material layer comprising collagen, proteoglycan and hyaluronic acid, and a second composite material layer comprising collagen and calcium phosphate, which are bonded to each other via a bonding layer comprising collagen, proteoglycan, hyaluronic acid and calcium phosphate.

2. The artificial bone-cartilage composite according to claim 1, wherein said first composite material layer comprises 15-95% by mass of collagen, 4.9-70% by mass of proteoglycan, and 0.1-20% by mass of hyaluronic acid.

3. The artificial bone-cartilage composite according to claim 1, wherein said second composite material layer is a porous apatite/collagen composite.

4. The artificial bone-cartilage composite according to claim 3, wherein an apatite/collagen ratio in said second composite material layer is 8/2-2/8 by mass.

5. The artificial bone-cartilage composite according to claim 1, which is cross-linked.

6. A method for producing an artificial bone-cartilage composite comprising a first composite material layer comprising collagen, proteoglycan and hyaluronic acid, and a second composite material layer comprising collagen and calcium phosphate, which are bonded to each other via a bonding layer comprising collagen, proteoglycan, hyaluronic acid and calcium phosphate, the method comprising the steps of (1) preparing a first composite material comprising collagen, proteoglycan and hyaluronic acid, (2) preparing a second composite material comprising collagen and calcium phosphate, (3) laminating said first composite material and said second composite material, and (4) freeze-drying the resultant laminate.

7. The method for producing an artificial bone-cartilage composite according to claim 6, wherein said first composite material and/or said second composite material are subject to a centrifugal treatment before or after the laminating step.

8. The method for producing an artificial bone-cartilage composite according to claim 6, wherein said laminate is gelled before the freeze-drying step.

9. The method for producing an artificial bone-cartilage composite according to claim 6, wherein said first composite material is in the form of gel, and said second composite material is in the form of slurry or gel.
